(19) United States
(12) Patent Application Publication
(10) Pub. No.: US 2006/0052404 A1
(43) Pub. Date: Mar. 9, 2006

(54) Pharmaceutical formulations comprising substituted xanthine compounds

(76) Inventors: Alfred R Rudolph, Los Altos Hills, CA (US); Cynthia W Tuthill, Menlo Park, CA (US)

Correspondence Address: HELLER EHRMAN LLP, 275 MIDDLEFIELD ROAD, MENLO PARK, CA, 94025-3506, US

(21) Appl. No.: 10/492,012

(22) PCT Filed: Oct. 3, 2002
(86) PCT No.: PCT/US02/31810

§ 371 Date: Oct. 20, 2005

(30) Foreign Application Priority Data
Oct. 26, 2001 (US) 60345892
Oct. 30, 2001 (US) 60335008

(51) Int. Cl. A61K 31/522

(52) U.S. Cl. 514/263.34

(57) ABSTRACT

The present invention provides novel pharmaceutical compositions comprising substituted xanthine compounds useful for the treatment of cystic fibrosis and other diseases, and methods of use thereof.

### BACKGROUND OF THE INVENTION

1. Field of the Invention

The present invention relates to novel pharmaceutical formulations. Specifically, the present invention provides novel formulations of substituted xanthine compounds for the treatment of cystic fibrosis, and other diseases, including chronic obstructive pulmonary diseases (COPDs).

2. Description of the Related Art

Cystic Fibrosis and COPD

Cystic fibrosis (CF) is the most common fatal genetic disease affecting the Caucasian population. The incidence of the disease among Caucasian Americans is approximately 1 of every 2500 live births. Among Afro-Americans, the incidence is less frequent, with about 1 of every 17,000 live births. An estimated 70,000 victims suffer from the disease worldwide. Apart from the loss of life and loss of quality of life, it costs about $50,000 a year to treat a cystic fibrosis patient in the United States, mostly using antibiotics, enzyme, and other drugs that help prolong life, but inevitably fail to save it.

Cystic fibrosis is a whole body disease, and the associated abnormalities are many and varied, due to the multi-systemic nature of the disease. Most of the diverse symptoms displayed are attributed to underlying abnormality in exocrine gland function. Three general types of pathophysiology are observed in the exocrine glands of cystic fibrosis patients. These are (1) glands become obstructed due to viscid or solid material in the luminal space of the gland (e.g., as observed in the pancreas and intestinal glands), (2) glands are histologically abnormal and produce an excess of secretions (e.g., tracheobronchial glands), and (3) glands are histologically normal, but secrete excessive sodium (Na⁺) and chloride (CF) ions (e.g., the sweat glands).

Signs of the disease can manifest from the time of birth, and can vary widely in their severity. Inevitably, all patients suffering from the disease develop chronic progressive disease of the respiratory system, characterized by accumulation of excessively viscous mucus secretion, airway plugging, and opportunistic bacterial infection in the airway. Although many organ systems are affected, approximately 90% of patients eventually succumb to pulmonary failure exacerbated by chronic infection. In the majority of cases, pancreatic dysfunction occurs, and hepatobiliary and genitourinary diseases, including infertility, are also manifested. Although survival of cystic fibrosis patients has improved in recent years, the median survival is still only about 30 years despite the development and implementation of intensive supportive and prophylactic treatment

The pulmonary complications of cystic fibrosis are one example of a larger category of diseases, namely, those diseases that result in chronic obstruction of the airway, which includes the alveoli, bronchi, bronchioles and upper airway, including the trachea. Collectively, these disorders are broadly termed chronic obstructive pulmonary disease (COPD), or synonymously, chronic obstructive airway disorders (COAD), regardless of disease etiology. COPD encompasses various diseases, all of which share the common pathology of airway obstruction. The diseases that can manifest as COPD's can include, for example, cystic fibrosis, chronic bronchitis, emphysema and asthma. Furthermore, patients displaying COPD pathology may have complex and overlapping etiologies, for example, in asthmatic bronchitis. Treatment for COPD often uses bronchodilator drugs, which may offer some relief to the patient, regardless of disease etiology. Anti-inflammatory agents, antibiotics and/or oxygen therapy are also appropriate for some COPD patients.

The CFTR Gene and Gene-Product

Cystic fibrosis disease is caused by mutations in the cystic fibrosis transmembrane regulator (CFTR) gene. The most common of these mutations, accounting for approximately 75% of mutant CFTR alleles, results in the deletion of a phenylalanine residue at position 508 (written ΔPhe⁵⁰⁸ or ΔF508). More than 900 different mutations have been identified in the remaining 25% of the mutant CFTR alleles (Kunzelmann and Nitschke, Exp. Nephrol., 8:332-342 [2000]).

The ΔF508 mutation commonly found in CFTR alleles is located within the first nucleotide binding fold (NBF-1) of the CFTR protein (Schoumacher et al., Proc. Natl. Acad. Sci., 87:4012-4016 [1990]; Riordan et al., Science 245:1066-1073 [1979]). More specifically, the ΔF508 mutation is located in a portion of the NBF-1, flanked on the N-terminal side by amino acid position 458-471 (known as the Walker A sequence) and on the C-terminal side by amino acid position 548-560 (known as the C-domain), and further by amino acid position 561-573 (known as the Walker B domain). The physiological function of the CFTR amino acids located between positions 471 and 561 is unknown.

The regulated movement of inorganic ions across the cell membrane is required to maintain a proper electrical potential across cellular membranes, as well as maintaining an appropriate intracellular ionic strength. For example, sodium (Na⁺), chloride (Cl⁻) ions, potassium (K⁺), and calcium (Ca²⁺) ions cross animal cell membranes in such a manner that K+ and Ca²⁺ are generally accumulated intracellularly, whereas Na⁺, in large measure, is excluded from the cell interior. The movement of these ions across the cell membrane is mediated by membrane-bound Na⁺/K⁺ and Ca²⁺-dependent ATPases. Conductance of chloride ions across the cell membrane is also actively regulated by at least one ion-specific chloride channel (Edwards, Neuroscience 7:1335-1366 [1982]), resulting in an under representation of intracellular Cl⁻ relative to the overall negative intracellular charge.

The wild-type 1480 amino acid CFTR protein appears to be part of a membrane-associated cAMP-regulated chloride transporter (i.e., a chloride channel) that actively secretes chloride (Cl⁻) ions across epithelial cell apical membranes from the cell interior to the cell exterior. Certain mutant forms of the CFTR protein, including CFTR-ΔF508, are defective in this process. Lack of function of the normal CFTR protein results in an abnormal charge potential across the apical surfaces of epithelial cell membranes due to reduced cellular chloride conductance. Thus, chloride, and consequently sodium, transport across epithelial membranes of an individual expressing a mutant CFTR-ΔF508 protein is abnormal. It is also known that cells expressing the mutant CFTR-ΔF508 protein demonstrate a higher than normal percentage of the protein bound to the endoplasmic reticulum compared to cells expressing wild-type CFTR protein, indicating an abrogation of CFTR trafficking, retention and degradation (Roomans, Exp. Opin. Invest Drugs 10(1):1-19 [2001]; Kunzelmann and Nitschke, Exp. Nephrol., 8:332-342 [2000]). This mutation and resulting ion conductance impairment as seen in cystic fibrosis patients is thought to be the cause of the cellular pathology observed in these patients, including the respiratory pathophysiology.

Use of Xanthine Compounds in the Treatment of Cystic Fibrosis and COPD

Various nucleotides, nucleotide derivatives, purine compounds, and most particularly, xanthine derivatives, show promise in stimulating chloride transport activity, and thus, are candidate therapeutic agents in the treatment of cystic fibrosis (Roomans, Exp. Opin. Invest. Drugs 10(1):1-19 [2001]; Rodgers and Knox, Eur. Respir. J., 17:1314-1321 [2001]). These xanthine compounds have a variety of advantageous activities, including acting as pulmonary vasodilators, bronchodilators and smooth muscle relaxants. In addition, some of these compounds also have other actions, including coronary vasodilator, diuretic, cardiac and cerebral stimulant and skeletal muscle stimulant (see, U.S. Pat. No. 5,032,593).

U.S. Pat. No. 4,548,818 describes the use of 3-alkyl-xanthines, such as 3-cyclopentyl-3,7-dihydro-1H-purine-2,6-dione, to treat chronic obstructive airway disease (COPD), as well as cardiac disease. Di-substituted forms of xanthine are disclosed as bronchodilatory agents. U.S. Pat. No. 5,032,593 describes the use of 1,3-alkyl substituted 8-phenyl-xanthine compounds, such as 1-n-propyl-3-methyl- and 1-methyl-3-n-propyl-substituted xanthine derivatives, in the treatment of bronchoconstriction.

U.S. Pat. No. 5,096,916 describes the use of imidazoline compounds in the treatment of COPD, including cystic fibrosis, chronic bronchitis and emphysema, or COPD in association with asthma. The compound tolazoline is the preferred vasodilator compound, although other useful compounds are also taught.

Historically, the substituted-xanthine compound theophylline has been administered to asthmatic and cystic fibrosis patients to enhance lung function. Other compounds resembling theophylline in basic structure have been identified which possess advantageous activities, including evoking chloride efflux from cystic fibrosis cells. These compounds include 1,3-dipropyl-8-cyclopentylxanthine (CPX). CPX (and its related xanthine amino congeners) is a potent A1 adenosine receptor antagonist that promotes chloride efflux from a human epithelial cell line expressing the CFTR-ΔF508 mutation (see, e.g., U.S. Pat. Nos. 5,366,977, 5,877,179 and 6,083,954, and Eidelman et al., Proc. Natl. Acad. Sci. USA, 89:5562-5566 [1992]; Guay-Broder et al., Biochemistry 34(28):9079-9087 [1995]; Jacobson et al., Biochemistry 34(28):9088-9094 [1995]; Arispe et al., Jour. Biol. Chem., 273(10):5727-5734 [1998]). Based on research that originated at the National Institutes of Health, SciClone Pharmaceuticals, Inc., California, U.S., is currently developing CPX as a promising new protein-repair therapy for cystic fibrosis treatment.

Compounds related in structure to CPX and activating chloride ion efflux in cells having the ΔF508 mutation, are also known, and have been suggested to have therapeutic value in the treatment of cystic fibrosis or other diseases. Such compounds include, for example, N,N-diallylcyclohexylxanthine (DAX; synonymously, 1,3-diallyl-8-cyclohexylxanthine, DCHX), 1,3-dipropyl-7-methylcyclopenthylxanthine (DP-CPX), cyclohexylcaffeine (CHC), and xanthine amino congener. See, e.g., U.S. Pat. Nos. 5,366,977, 5,877,179 and 6,083,954.

Accordingly, xanthine-derivatives are promising therapeutic agents for the treatment of cystic fibrosis and other chronic obstructive airway disorders. A prerequisite of successful therapeutic application is, however, the development of stable pharmaceutical formulations, preferably for oral delivery, that provide good absorption and bioavailability, have suitable pharmacokinetic properties, and enable safe administration of the therapeutically active compounds. The present invention meets this need by providing stable, oil-based suspensions of therapeutically effective xanthine compounds. These formulations have excellent oral bioavailability and sufficient plasma half life for successful use in human therapy.

These and other objects and advantages of the present invention, as well as additional inventive features, will be apparent from the description of the invention provided herein.

### SUMMARY OF THE INVENTION

The invention relates to novel formulations of substituted xanthine compounds, where the formulations are liquid formulations suitable for oral delivery. These formulations comprise at least one substituted xanthine compound and a pharmaceutically acceptable oil. The invention also provides methods employing these novel formulations.

In one embodiment, the invention provides a liquid pharmaceutical formulation suitable for oral administration comprising an effective amount of a therapeutically active xanthine derivative, or a pharmaceutically acceptable salt thereof, in admixture with a pharmaceutically acceptable oil. In some embodiments, the xanthine derivative is hydrophobic. In other embodiments, the formulation is a solution, while in other embodiments, the formulation is a suspension. Where the formulation is a suspension, the xanthine derivative or a pharmaceutically acceptable salt thereof, can be in the form of particles, and the particles optionally have a mean diameter less than about 100 microns. In some embodiments comprising a suspension, the suspension is substantially homogenous.

In some embodiments, the oil in the formulation is a vegetable oil. In some embodiments, the vegetable oil can be corn oil, almond oil, coconut oil, cottonseed oil, mustard seed oil, olive oil, palm oil, peanut oil, safflower oil, sesame oil, soybean oil, sunflower oil, and partially or fully hydrogenated derivatives of said oils. In one embodiment, corn oil is the vegetable oil.

In another embodiment, the invention provides a suspension suitable for oral administration comprising, as active ingredient, an effective amount of a substituted xanthine compound. It is not intended that the invention be limited to the use of any particular substituted xanthine compound or compounds. In this embodiment, the dispersed active ingredient is in the form of particles having a mean diameter less than about 100 microns.

In a related embodiment, the invention provides a suspension suitable for oral administration comprising, as active ingredient, an effective amount of a substituted xanthine compound. In this embodiment, the substituted xanthine has the formula 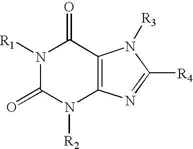
(I), wherein
R1 and R2 are the same or different and are C(1-6)alkyl or C(1-6)alkenyl, or hydrogen; R3 is C(1-6)alkyl or hydrogen, and R4 is C(4-8)cycloalkyl, aryl or hydrogen, wherein at least one of R1, R2 and R3 is other than hydrogen, or a therapeutically active derivative thereof, or a pharmaceutically acceptable salt of said substituted xanthine, or a pharmaceutically acceptable salt of said therapeutically active derivative. Furthermore in this embodiment, the active ingredient is in the form of particles having a mean diameter less than about 100 microns, and the particles are dispersed in a pharmaceutically acceptable oil. In some embodiments, the substituted xanthine is hydrophobic. In other embodiments, the suspension is substantially homogenous.

In various embodiments of the suspension formulation, alternatively at least 70%, or at least 80%, or at least 90%, or substantially all of the particles in the suspension have a diameter less than about 100 microns.

In some embodiments, the suspension formulation can comprise a pharmaceutically acceptable preservative, a pharmaceutically acceptable antioxidant, or both.

In various embodiments of the suspension formulation, the structure of the substituted xanthine is defined. For example, in one embodiment, in reference to formula (I), R1 and R2 are the same or different and are C(1-6)alkyl or C(1-6)alkenyl; R3 is C(1-6)alkyl or hydrogen, and R4 is C(4-8) cycloalkyl. In another embodiment, R1 and R2 are the same and are methyl or allyl, R3 is ethyl, cyclopropylmethyl or hydrogen, and R4 is cyclohexyl, provided that R1 is allyl when R3 is hydrogen, and R1 is methyl when R3 is ethyl or cyclopropylmethyl. In another embodiment, R1 and R2 are both methyl, R3 is ethyl, cyclopropylmethyl, and R4 is cyclohexyl. In another embodiment, R1 and R2 are allyl, R3 is hydrogen, and R4 is cyclohexyl, cyclohexylmethyl, or cycloheptyl. In another embodiment, R1 is methyl, R2 is allyl, R3 is cyclopropylmethyl or ethyl, and R4 is cyclohexyl. In yet another embodiment, R1 and R2 are the same or different, and are methyl, propyl, allyl or hydrogen; R3 is methyl or hydrogen, and R4 is cyclohexyl or cyclopentyl. In some embodiments, the substituted xanthine compound is further defined, and can be 1,3-dipropyl-8-cyclopentylxanthine (CPX), 1,3-diallyl-cyclohexylxanthine (DAX/DCHX), 1,3-dipropyl-7-methylcyclopenthylxanthine (DP-CPX), cyclohexylcaffeine (CHC), or xanthine amino congener (XAC). In one preferred embodiment, the substituted xanthine is 1,3-dipropyl-8-cyclopentylxanthine (CPX).

The invention also provides methods for the activation of ion efflux in ion efflux deficient cells. In this method, the deficient cells are contacted, directly or indirectly, with an effective amount of a liquid suspension suitable for oral administration, where the suspension comprises an effective amount of a therapeutic active ingredient, wherein said active ingredient is a substituted xanthine. Furthermore, the active ingredient is in the form of particles having a mean diameter less than about 100 microns, and the particles are in admixture with a pharmaceutically acceptable oil. It is not intended that the substituted xanthine used be particularly limited, as use of any therapeutically active substituted xanthine compound, derivative of any such compound, or pharmaceutically acceptable salt of any such xanthine compound, is within the scope of the invention.

In a related method provided by the invention for the activation of ion efflux in ion efflux deficient cells, the substituted xanthine compound is generally defined. In this method, the deficient cells are contacted, directly or indirectly, with an effective amount of a liquid suspension suitable for oral administration, where the suspension comprises an effective amount of a therapeutic active ingredient, wherein said active ingredient is a substituted xanthine. Furthermore, the active ingredient is in the form of particles having a mean diameter less than about 100 microns, and the particles are in admixture with a pharmaceutically acceptable oil. In this method, the substituted xanthine is generally defined by the formula: 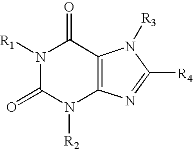
(I), wherein
R1 and R2 are the same or different and are C(1-6)alkyl or C(1-6)alkenyl, or hydrogen; R3 is C(1-6)alkyl or hydrogen, and R4 is C(4-8)cycloalkyl, aryl or hydrogen, wherein at least one of R1, R2 and R3 is other than hydrogen. Also encompassed by this method is use of therapeutically active derivatives of the substituted xanthine, pharmaceutically acceptable salt of the substituted xanthine, and pharmaceutically acceptable salt of the therapeutically active derivative. In some embodiments, the substituted xanthine compound is further defined, and can be 1,3-dipropyl-8-cyclopentylxanthine (CPX), 1,3-diallyl-cyclohexylxanthine (DAX/DCHX), 1,3-dipropyl-7-methylcyclopenthylxanthine (DP-CPX), cyclohexylcaffeine (CHC), or xanthine amino congener (XAC). In one preferred embodiment, the substituted xanthine is 1,3-dipropyl-cyclopentylxanthine (CPX).

In some embodiments of this methods, cells to be treated are cystic fibrosis (CF) cells, and in other embodiments, the CF cells have a CFTR-ΔF508 mutation.

In some embodiments, the pharmaceutically acceptable oil in the formulation is a vegetable oil. In some embodiments, the vegetable oil can be corn oil, almond oil, coconut oil, cottonseed oil, mustard seed oil, olive oil, palm oil, peanut oil, safflower oil, sesame oil, soybean oil, sunflower oil, and partially or fully hydrogenated derivatives of said oils.

The invention also provides methods for the activation of ion efflux in ion efflux deficient cells. In this method, the deficient cells are contacted, directly or indirectly, with an effective amount of a liquid suspension suitable for oral administration, where the suspension comprises an effective amount of a therapeutic active ingredient, wherein said active ingredient is a substituted xanthine. Furthermore, the active ingredient is in the form of particles having a mean diameter less than about 100 microns, and the particles are in admixture with a pharmaceutically acceptable oil. It is not intended that the substituted xanthine used be particularly limited, as use of any therapeutically active substituted xanthine compound, derivative of any such compound, or pharmaceutically acceptable salt of any such xanthine compound, is within the scope of the invention.

In a related method provided by the invention for the activation of ion efflux in ion efflux deficient cells, the substituted xanthine compound is generally defined. In this method, the deficient cells are contacted, directly or indirectly, with an effective amount of a liquid suspension suitable for oral administration, where the suspension comprises an effective amount of a therapeutic active ingredient, wherein said active ingredient is a substituted xanthine. Furthermore, the active ingredient is in the form of particles having a mean diameter less than about 100 microns, and the particles are in admixture with a pharmaceutically acceptable oil. In this method, the substituted xanthine is generally defined by the formula: 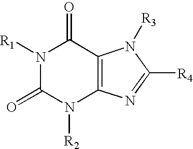
(I), wherein
R1 and R2 are the same or different and are C(1-6)alkyl or C(1-6)alkenyl, or hydrogen; R3 is C(1-6)alkyl or hydrogen, and R4 is C(4-8)cycloalkyl, aryl or hydrogen, wherein at least one of R1, R2 and R3 is other than hydrogen. Also encompassed by this method is use of therapeutically active derivatives of the substituted xanthine, pharmaceutically acceptable salt of the substituted xanthine, and pharmaceutically acceptable salt of the therapeutically active derivative. In some embodiments, the substituted xanthine compound is further defined, and can be 1,3-dipropyl-8-cyclopentylxanthine (CPX), 1,3-diallyl-cyclohexylxanthine (DAX/DCHX), 1,3-dipropyl-7-methylcyclopenthylxanthine (DP-CPX), cyclohexylcaffeine (CHC), or xanthine amino congener (XAC). In one preferred embodiment, the substituted xanthine is 1,3-dipropyl-8-cyclopentylxanthine (CPX).

In some embodiments of this methods, cells to be treated are cystic fibrosis (CF) cells, and in other embodiments, the CF cells have a CFTR-ΔF508 mutation.

In some embodiments, the pharmaceutically acceptable oil in the formulation is a vegetable oil. In some embodiments, the vegetable oil can be corn oil, almond oil, coconut oil, cottonseed oil, mustard seed oil, olive oil, palm oil, peanut oil, safflower oil, sesame oil, soybean oil, sunflower oil, and partially or fully hydrogenated derivatives of said oils.

The invention also provides methods for the activation of ion efflux in ion efflux deficient cells. In this method, the deficient cells are contacted, directly or indirectly, with an effective amount of a liquid suspension suitable for oral administration, where the suspension comprises an effective amount of a therapeutic active ingredient, wherein said active ingredient is a substituted xanthine. Furthermore, the active ingredient is in the form of particles having a mean diameter less than about 100 microns, and the particles are in admixture with a pharmaceutically acceptable oil. It is not intended that the substituted xanthine used be particularly limited, as use of any therapeutically active substituted xanthine compound, derivative of any such compound, or pharmaceutically acceptable salt of any such xanthine compound, is within the scope of the invention.

In a related method provided by the invention for the activation of ion efflux in ion efflux deficient cells, the substituted xanthine compound is generally defined. In this method, the deficient cells are contacted, directly or indirectly, with an effective amount of a liquid suspension suitable for oral administration, where the suspension comprises an effective amount of a therapeutic active ingredient, wherein said active ingredient is a substituted xanthine. Furthermore, the active ingredient is in the form of particles having a mean diameter less than about 100 microns, and the particles are in admixture with a pharmaceutically acceptable oil. In this method, the substituted xanthine is generally defined by the formula: 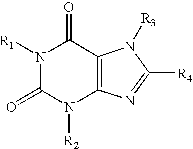
(I), wherein
R1 and R2 are the same or different and are C(1-6)alkyl or C(1-6)alkenyl, or hydrogen; R3 is C(1-6)alkyl or hydrogen, and R4 is C(4-8)cycloalkyl, aryl or hydrogen, wherein at least one of R1, R2 and R3 is other than hydrogen. Also encompassed by this method is use of therapeutically active derivatives of the substituted xanthine, pharmaceutically acceptable salt of the substituted xanthine, and pharmaceutically acceptable salt of the therapeutically active derivative. In some embodiments, the substituted xanthine compound is further defined, and can be 1,3-dipropyl-8-cyclopentylxanthine (CPX), 1,3-diallyl-cyclohexylxanthine (DAX/DCHX), 1,3-dipropyl-7-methylcyclopenthylxanthine (DP-CPX), cyclohexylcaffeine (CHC), or xanthine amino congener (XAC). In one preferred embodiment, the substituted xanthine is 1,3-dipropyl-8-cyclopentylxanthine (CPX).

In some embodiments of this methods, cells to be treated are cystic fibrosis (CF) cells, and in other embodiments, the CF cells have a CFTR-ΔF508 mutation.

In some embodiments, the pharmaceutically acceptable oil in the formulation is a vegetable oil. In some embodiments, the vegetable oil can be corn oil, almond oil, coconut oil, cottonseed oil, mustard seed oil, olive oil, palm oil, peanut oil, safflower oil, sesame oil, soybean oil, sunflower oil, and partially or fully hydrogenated derivatives of said oils.

The invention also provides methods for the activation of ion efflux in ion efflux deficient cells. In this method, the deficient cells are contacted, directly or indirectly, with an effective amount of a liquid suspension suitable for oral administration, where the suspension comprises an effective amount of a therapeutic active ingredient, wherein said active ingredient is a substituted xanthine. Furthermore, the active ingredient is in the form of particles having a mean diameter less than about 100 microns, and the particles are in admixture with a pharmaceutically acceptable oil. It is not intended that the substituted xanthine used be particularly limited, as use of any therapeutically active substituted xanthine compound, derivative of any such compound, or pharmaceutically acceptable salt of any such xanthine compound, is within the scope of the invention.

In a related method provided by the invention for the activation of ion efflux in ion efflux deficient cells, the substituted xanthine compound is generally defined. In this method, the deficient cells are contacted, directly or indirectly, with an effective amount of a liquid suspension suitable for oral administration, where the suspension comprises an effective amount of a therapeutic active ingredient, wherein said active ingredient is a substituted xanthine. Furthermore, the active ingredient is in the form of particles having a mean diameter less than about 100 microns, and the particles are in admixture with a pharmaceutically acceptable oil. In this method, the substituted xanthine is generally defined by the formula: 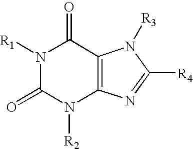
(I), wherein
R1 and R2 are the same or different and are C(1-6)alkyl or C(1-6)alkenyl, or hydrogen; R3 is C(1-6)alkyl or hydrogen, and R4 is C(4-8)cycloalkyl, aryl or hydrogen, wherein at least one of R1, R2 and R3 is other than hydrogen. Also encompassed by this method is use of therapeutically active derivatives of the substituted xanthine, pharmaceutically acceptable salt of the substituted xanthine, and pharmaceutically acceptable salt of the therapeutically active derivative. In some embodiments, the substituted xanthine compound is further defined, and can be 1,3-dipropyl-8-cyclopentylxanthine (CPX), 1,3-diallyl-cyclohexylxanthine (DAX/DCHX), 1,3-dipropyl-7-methylcyclopenthylxanthine (DP-CPX), cyclohexylcaffeine (CHC), or xanthine amino congener (XAC). In one preferred embodiment, the substituted xanthine is 1,3-dipropyl-8-cyclopentylxanthine (CPX).

In some embodiments of this methods, cells to be treated are cystic fibrosis (CF) cells, and in other embodiments, the CF cells have a CFTR-ΔF508 mutation.

In some embodiments, the pharmaceutically acceptable oil in the formulation is a vegetable oil. In some embodiments, the vegetable oil can be corn oil, almond oil, coconut oil, cottonseed oil, mustard seed oil, olive oil, palm oil, peanut oil, safflower oil, sesame oil, soybean oil, sunflower oil, and partially or fully hydrogenated derivatives of said oils.

The invention also provides methods for the treatment of a disease or condition characterized by defective ion transport associated with reduced or abnormal CFTR activity. In this method, a subject in need is administered a therapeutically effective amount of a liquid formulation suitable for oral administration, where the formulation comprises an effective amount of a therapeutic active ingredient, where the active ingredient is a substituted xanthine. Furthermore, the active ingredient is in the form of particles having a mean diameter less than about 100 microns, and the particles are in admixture with a pharmaceutically acceptable oil. It is not intended that the substituted xanthine used be particularly limited, as use of any therapeutically active substituted xanthine compound, derivative of any such compound, or pharmaceutically acceptable salt of any such xanthine compound, is within the scope of the invention.

In a related method provided by the invention for the treatment of a disease or condition characterized by defective ion transport associated with reduced or abnormal CFTR activity, the substituted xanthine compound is generally defined. In this method, a subject in need is administered a therapeutically effective amount of a liquid formulation suitable for oral administration, where the formulation comprises an effective amount of a therapeutic active ingredient, where the active ingredient is a substituted xanthine. Furthermore, the active ingredient is in the form of particles having a mean diameter less than about 100 microns, and the particles are in admixture with a pharmaceutically acceptable oil. In this method, the substituted xanthine is generally defined by the formula: 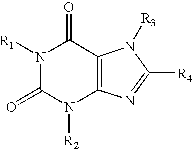
(I), wherein
R1 and R2 are the same or different and are C(1-6)alkyl or C(1-6)alkenyl, or hydrogen; R3 is C(1-6)alkyl or hydrogen, and R4 is C(4-8)cycloalkyl, aryl or hydrogen, wherein at least one of R1, R2 and R3 is other than hydrogen. Also encompassed by this method is use of therapeutically active derivatives of the substituted xanthine, pharmaceutically acceptable salt of the substituted xanthine, and pharmaceutically acceptable salt of the therapeutically active derivative. In some embodiments, the substituted xanthine compound is further defined, and can be 1,3-dipropyl-8-cyclopentylxanthine (CPX), 1,3-diallyl-cyclohexylxanthine (DAX/DCHX), 1,3-dipropyl-7-methylcyclopenthylxanthine (DP-CPX, cyclohexylcaffeine (CHC), or xanthine amino congener (XAC). In one preferred embodiment, the substituted xanthine is 1,3-dipropyl-8-cyclopentylxanthine (CPX).

In some embodiments of this methods, the disease or condition to be treated is a chronic obstructive airway disorder. In another embodiment, the disease or condition to be treated is cystic fibrosis.

In some embodiments, the pharmaceutically acceptable oil in the formulation is a vegetable oil. In some embodiments, the vegetable oil can be corn oil, almond oil, coconut oil, cottonseed oil, mustard seed oil, olive oil, palm oil, peanut oil, safflower oil, sesame oil, soybean oil, sunflower oil, and partially or fully hydrogenated derivatives of said oils.

The invention also provides methods for the treatment of a disease or condition characterized by chronic airway obstruction. In this method, a subject in need is administered a therapeutically effective amount of a liquid formulation suitable for oral administration, where the formulation comprises an effective amount of a therapeutic active ingredient, where the active ingredient is a substituted xanthine. Furthermore, the active ingredient is in the form of particles having a mean diameter less than about 100 microns, and the particles are in admixture with a pharmaceutically acceptable oil. It is not intended that the substituted xanthine used be particularly limited, as use of any therapeutically active substituted xanthine compound, derivative of any such compound, or pharmaceutically acceptable salt of any such xanthine compound, is within the scope of the invention.

In a related method provided by the invention for the treatment of a disease or condition characterized by chronic airway obstruction, the substituted xanthine compound is generally defined. In this method, a subject in need is administered a therapeutically effective amount of a liquid formulation suitable for oral administration, where the formulation comprises an effective amount of a therapeutic active ingredient, where the active ingredient is a substituted xanthine. Furthermore, the active ingredient is in the form of particles having a mean diameter less than about 100 microns, and the particles are in admixture with a pharmaceutically acceptable oil. In this method, the substituted xanthine is generally defined by the formula: 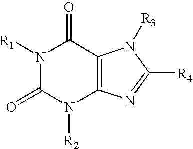
(I), wherein
R1 and R2 are the same or different and are C(1-6)alkyl or C(1-6)alkenyl, or hydrogen; R3 is C(1-6)alkyl or hydrogen, and R4 is C(4-8)cycloalkyl, aryl or hydrogen, wherein at least one of R1, R2 and R3 is other than hydrogen. Also encompassed by this method is use of therapeutically active derivatives of the substituted xanthine, pharmaceutically acceptable salt of the substituted xanthine, and pharmaceutically acceptable salt of the therapeutically active derivative.

The present invention also provides articles of manufacture comprising the formulations of the invention. In one embodiment, the article of manufacture provides a container, a liquid pharmaceutical formulation suitable for oral administration, comprising a therapeutic active ingredient, wherein said active ingredient is a substituted xanthine, and directions for the administration of the formulation for the treatment of a disease or condition characterized by defective ion transport associated with reduced or abnormal CTFR activity. Furthermore, the active ingredient is in the form of particles having a mean diameter less than about 100 microns, and the particles are in admixture with a pharmaceutically acceptable oil. It is not intended that the substituted xanthine used in the article of manufacture be particularly limited, as use of any therapeutically active substituted xanthine compound, derivative of any such compound, or pharmaceutically acceptable salt of any such xanthine compound, is within the scope of the invention.

In a related composition, the present invention also provides articles of manufacture comprising the formulations of the invention as described above, and the substituted xanthine compound is generally defined. In one embodiment, the article of manufacture provides a container, a liquid pharmaceutical formulation suitable for oral administration, comprising a therapeutic active ingredient, wherein said active ingredient is a substituted xanthine, and directions for the administration of the formulation for the treatment of a disease or condition characterized by defective ion transport associated with reduced or abnormal CTFR activity. Furthermore, the active ingredient is in the form of particles having a mean diameter less than about 100 microns, and the particles are in admixture with a pharmaceutically acceptable oil. In this embodiment, the substituted xanthine is generally defined by the formula: 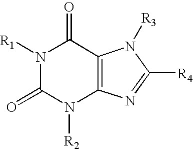
(I), wherein
R1 and R2 are the same or different and are C(1-6)alkyl or C(1-6)alkenyl, or hydrogen; R3 is C(1-6)alkyl or hydrogen, and R4 is C(4-8)cycloalkyl, aryl or hydrogen, wherein at least one of R1, R2 and R3 is other than hydrogen, or a therapeutically active derivative thereof, or a pharmaceutically acceptable salt of said substituted xanthine, or a pharmaceutically acceptable salt of said therapeutically active derivative.

In other embodiments of the article of manufacture, the instructions are in the form of a package insert. In other embodiments, the disease or condition to be treated is cystic fibrosis.

In another embodiment of the article of manufacture, the container is a bottle. In another embodiment, the bottle is a glass bottle. In still another embodiment, the glass bottle is secured by a cap.

### BRIEF DESCRIPTION OF THE DRAWINGS

FIG. 1 shows a graph of rat plasma CPX concentration (ng/ml) as a function of time (in hours) using two different drug formulations. The CPX concentration was measured at regular time intervals following oral administration of an approximately 100 mg dose, where the dose was delivered in either a corn oil formulation (dark line) or a water formulation (light line).

FIG. 2 shows a table of CPX concentrations in the blood plasma of four male Beagle dog at regular time intervals (shown in hours) following the administration single oral doses 30 mg/kg of various CPX formulations. These formulations were xanthan gum (homogenized), sodium carboxymethylcellulose [NaCMC] (homogenized), sodium carboxymethylcellulose [NaCMC] (non-homogenized), and corn oil (homogenized).

FIG. 3 shows a graphical representation of the data provided in FIG. 3, where the mean CPX plasma concentration (ng/ml) of each dog treatment group is plotted against time (in hours), for each drug formulation. This representation plots the mean plasma CPX concentrations on a linear axis.

FIG. 4 shows a graphical representation of the data provided in FIG. 3, where the mean CPX plasma concentration (ng/ml) of each dog treatment group is plotted against time (in hours), for each drug formulation. This representation plots the mean plasma CPX concentrations on a semilogarithmic axis.

FIG. 5 shows human blood plasma CPX concentrations at regular time intervals (in hours) following administration of a single 300 mg oral dose of CPX. Data for two human subject groups is shown, one group (n=3) receiving the CPX dose in a corn oil formulation (diamonds), and the other group (n=4) receiving the CPX dose in a hard gelatin capsule (triangles). Standard error of the mean for each time point is shown as a vertical line.

### DETAILED DESCRIPTION OF THE INVENTION

Definitions

Unless defined otherwise, technical and scientific terms used herein have the same meaning as commonly understood by one of ordinary skill in the art to which this invention belongs. For further information see, for example, Comprehensive Organic Chemistry, I. O. Sutherland editor, Pergamon Press, 1979; Vogel's Textbook of Practical Organic Chemistry, 5th Ed., 1989; Van Nostrand Reinhold, Encyclopedia of Chemistry, 4th Ed., 1984; John McMurry, Organic Chemistry, 5th Ed., 2000; Vollhardt and Schore, Organic Chemistry, W.H. Freeman and Co., New York, 1995. One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “suspension” is used for its ordinary meaning to describe a dispersion of solid particles in a liquid. Thus, an “oil-based suspension” means the suspension of solid particles in an oil.

The term “homogenized” or “homogenous” is used to refer to a substantially uniform distribution of solid particles (e.g., drug particles) in a suspension, such as an oil-based suspension.

The term “liquid formulation” is used to describe any mixture of two or more substances which is substantially liquid in character. Liquid formulations include, without limitation, solutions, suspensions and dispersions of an active ingredient, and optionally further components, in a liquid excipient, preferably an oil, such as a vegetable oil. Liquid formulations, as defined herein, can comprise both particulate and dissolved components. Furthermore, the liquid formulations herein can comprise the same component in both particulate and dissolved form.

“Particle size distribution” means the number of particles in individual size classes divided by the total number of particles in a sample, expressed as percentages. Particle size distribution can be determined by a variety of techniques known in the art, such as quantitative microscopic examination, or laser diffraction methodology. A preferred method is laser diffraction analysis (also called low angle light scattering), by which dry powders can be measured directly and liquid suspensions and emulsions can be measured in a re-circulating cell. This gives high reproducibility and enables the use of dispersing agents and surfactants for the determination of primary particle size. Particle size analyzers are commercially available, for example from Beckman Coulter, U.S., Laval Lab Inc., Canada, and Malvern Instruments Ltd., USA, the manufacturer of a variety of Mastersizer® particle analyzers.

A suspension in which “substantially all” particles has a diameter less than 100 microns contains at least about 95%, more preferably at least about 98%, even more preferably at least about 99%, most preferably at least about 99.5% particles with a diameter less than about 100 microns.

The “pharmaceutically acceptable oil” can be any natural or synthetic vegetable or animal oil suitable for pharmaceutical use, comprising mono-, di-, or triglyceryl esters of saturated and/or unsaturated fatty acids, alone or in combination.

The term “mammal” or “mammalian species” refers to any animal classified as a mammal, including humans, domestic and farm animals, and zoo, sports, or pet animals, such as dogs, cats, cattle, horses, sheep, pigs, goats, rabbits, as well as rodents such as mice and rats, etc. Preferably, the mammal is human.

The terms “subject” or “patient,” as used herein, are used interchangeably, and can refer to any to animal, and preferably a mammal, that is the subject of an examination, treatment, analysis, test or diagnosis. In one embodiment, humans are a preferred subject. A subject or patient may or may not have a disease or other pathological condition.

The terms “disease,” “disorder” and “condition” are used interchangeably herein, and refer to any disruption of normal body function, or the appearance of any type of pathology. The etiological agent causing the disruption of normal physiology may or may not be known. Furthermore, although two patents may be diagnosed with the same disorder, the particular symptoms displayed by those individuals may or may not be identical.

The terms “treat” or “treatment” refer to both therapeutic treatment and prophylactic or preventative measures, wherein the objective is to prevent or slow down (lessen) an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Those in need of treatment include those already with the condition or disorder as well as those prone to have the condition or disorder or those in which the condition or disorder is to be prevented.

“Chronic” administration refers to administration of the agent(s) in a continuous mode as opposed to an acute mode, so as to maintain a desired effect or level of agent(s) for an extended period of time.

“Intermittent” administration is treatment that is not consecutively done without interruption, but rather is periodic in nature.

Administration “in combination with” one or more further therapeutic agents includes simultaneous (concurrent) and consecutive administration in any order.

An “effective amount” is an amount sufficient to effect beneficial or desired therapeutic (including preventative) results. An effective amount can be administered in one or more administrations.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The term “alkyl” refers to a monovalent alkane (hydrocarbon) derived radical containing from 1 to 10 carbon atoms unless otherwise defined. It may be straight- or branched-chained, or cyclic. Preferred straight- or branched-chained alkyl groups include methyl, ethyl, propyl, isopropyl, butyl, and t-butyl. Preferred cycloalkyl groups include cyclopropyl, cyclobutyl, cycloheptyl, cyclopentyl, and cyclohexyl. The term “lower alkyl” refers to alkyl groups as hereinabove defined, having 1 to 6 carbon atoms. The term “alkyl” as used herein includes substituted alkyls.

The term “substituted alkyl” refers to alkyl as defined above, including one or more functional groups such as lower alkyl, aryl, acyl, halogen, hydroxy, amino, alkoxy, alkylamine, acylamino, acyloxy, aryloxy, aryloxyalkyl, mercapto, both saturated and unsaturated cyclic hydrocarbons, heterocycles, and the like. These groups may be attached to any carbon of the alkyl moiety.

The term “aryl” is used herein to refer to an aromatic substituent which may be a single aromatic ring or multiple aromatic rings which are fused together, linked covalently, or linked to a common group such as a methylene or ethylene moiety. The common linking group may also be a carbonyl as in benzophenone. The aromatic ring(s) may include phenyl, naphthyl, biphenyl, diphenylmethyl and benzophenone among others. The term “aryl” encompasses “arylalkyl,” “arylalkenyl,” and “arylalkinly.” The term “aryl” as used herein also includes substituted aryl.

“Substituted aryl” refers to aryl, as defined above, including one or more functional groups such as lower alkyl, acyl, halogen, alkylhalo, hydroxy, amino, alkoxy, alkylamine, acylamino, acyloxy, mercapto and both saturated and unsaturated cyclic hydrocarbons which are fused to the aromatic ring(s), linked covalently or linked to a common group such as a methylene or ethylene moiety. The linking group may also be a carbonyl such as in cyclohexyl phenyl ketone. The term “substituted aryl” encompasses “substituted arylalkyl.”

The term “aralkyl” or “arylalkyl” is used to refer to an aryl or heteroaryl moiety, as defined herein, attached through a C1-6 alkyl linker, where alkyl is as defined above.

The term “alkoxy” refers to a substituent with a straight- or branched-chain alkyl, alkenyl, or alkinyl group of the designated length, which is attached via an oxygen molecule. Representative alkoxy groups are methoxy, ethoxy, propoxy, isopropoxy, butoxy, isobutoxy, t-butoxy, pentoxy, isopentoxy, hexoxy, isohexoxy, allyloxy, propargyloxy, vinyloxy, etc.

The term “halogen” is used herein to refer to fluorine, bromine, chlorine and iodine atoms.

The term “amino” is used to refer to the group —NRR′, where R and R′ may independently be hydrogen, alkyl, substituted alkyl, aryl, substituted aryl or acyl.

The term “alkoxy” is used herein to refer to an —OR group, where R is a lower alkyl, substituted lower alkyl, aryl, substituted aryl, arylalkyl or substituted arylalkyl wherein the alkyl, aryl, substituted aryl, arylalkyl and substituted arylalkyl groups are as described herein. Suitable alkoxy radicals include, for example, methoxy, ethoxy, phenoxy, substituted phenoxy, benzyloxy, phenethyloxy, t-butoxy, etc.

The term “alkenyl” is used herein to refer to an unsaturated straight- or branched-chained, or cyclic monovalent hydrocarbon radical having at least one carbon-carbon double bond. The radical can be in either the cis or trans conformation about the double bond(s). Suitable alkenyl radicals include, for example, ethenyl, propenyl, isopropenyl, cyclopropenyl, butenyl, isobutenyl, cyclobutenyl, tert-butenyl, pentenyl, hexenyl, etc.

The term “pharmaceutically acceptable salt” refers to those salts of compounds which retain the biological effectiveness and properties of the free bases and which are obtained by reaction with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutically acceptable salts include, for example, alkali metal salts, such as sodium and potassium, alkaline earth metal salts and ammonium salts.

### DETAILED DESCRIPTION OF PREFERRED EMBODIMENTS OF THE INVENTION

The present invention provides novel, improved drug formulations comprising xanthine derivatives, where the novel formulations have improved characteristics such as drug uptake and bioavailability. These novel formulations find use in the treatment of various diseases, including but not limited to diseases resulting from defective ion transport due to reduced or abnormal CFTR activity, such as in cystic fibrosis, and also more broadly in the treatment of COPD. It is also contemplated that these formulations find use in the treatment of other diseases resulting from or exacerbated by improper-ion balances.

Xanthine Compounds Finding Use in the Treatment of Cystic Fibrosis or Other Diseases

Numerous xanthine derivatives are known to have properties consistent with therapeutic value in the treatment of cystic fibrosis and other diseases. Such xanthine derivatives include those characterized by the following general formula (I) 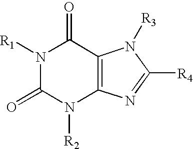
wherein R1 and R2 are the same or different and are C(1-6)alkyl, C(1-6)alkenyl or hydrogen; R3 is C(1-6)alkyl or hydrogen, and R4 is C(4-8)cycloalkyl, aryl or hydrogen, wherein at least one of R1, R2 and R3 is other than hydrogen, and therapeutically active derivatives thereof, or pharmaceutically acceptable salts of such compounds or their derivatives.

In a preferred embodiment, R1 and R2 are the same or different and are C(1-6)alkyl or C(1-6)alkenyl; R3 is C(1-6)alkyl or hydrogen, and R4 is C(4-8) cycloalkyl.

In another preferred embodiment, R1 and R2 are the same and are methyl or allyl, R3 is ethyl, cyclopropylmethyl or hydrogen, and R4 is cyclohexyl, provided that R1 is allyl when R3 is hydrogen, and R1 is methyl when R3 is ethyl or cyclopropylmethyl.

In yet another preferred embodiment, the formulation comprises a compound of formula (I) in which R1 and R2 are both methyl, R3 is ethyl or cyclopropylmethyl, and R4 is cyclohexyl.

In other preferred compounds, R1 and R2 are allyl, R3 is hydrogen; and R4 is cyclohexyl, cyclohexylmethyl, or cycloheptyl; or R1 is methyl, R2 is allyl, R3 is cyclopropylmethyl or ethyl, and R4 is cyclohexyl.

In still other preferred embodiments, the formulation comprises a compound of formula (I), wherein R1 and R2 are the same or different and are methyl, propyl, allyl or hydrogen; R3 is methyl or hydrogen, and R4 is cyclohexyl or cyclopentyl, and therapeutically active derivatives thereof, or pharmaceutically acceptable salts of such compounds or their derivatives.

The xanthine derivatives used in the formulations of the present invention can be synthesized by standard methods of organic chemistry, such as those described in the textbooks referenced above, and also e.g., in Jacobson et al., Biochemistry 34:9088-94 (1995); and U.S. Pat. Nos. 6,248,746; 6,180,791; 5,981,535; 5,366,977, 5,877,179 and 6,083,954. Alternatively, the compounds are commercially available (e.g., from Research Biochemicals International [RBI/Sigma], Natick, Me., and Sigma-Aldrich, St. Louis, Mo.).

Assays to identify xanthine derivatives, others than specifically disclosed herein, potentially useful for the treatment of cystic fibrosis and other diseases associated with reduced apical Cl—conductance in cells, are known in the art. For example, known drug screening assays for the identification of further useful xanthine derivatives include:

(A) Chloride Efflux Assay using Recombinant CFTR—Normal cultured mammalian cells, and most preferably human cells, are transfected with an expression vector encoding the wild-type or ΔF508 CFTR gene product. While in culture, the cells are treated with drug candidate compounds, and the chloride efflux across the cell membranes is measured, for example, by radiolabelled chloride isotopic equilibrium. Alternatively, changes in the osmolarity of the cell external medium can also be measured using an osmometer. This technique (or variations thereof) are described in various sources, such as but not limited to, U.S. Pat. No. 6,083,954; and Eidelman et al., Proc. Nail. Aced Sc. USA 89:5562-5566 [1992]. Compounds that stimulate chloride efflux in vitro are candidate drugs for further development and testing.

(B) Chloride Efflux Assay using Native Mutant CFTR—Similar to the technique described above, cultured mammalian cells, and most preferably, human cells derived from a cystic fibrosis patient (i.e., primary explant cultures), and most preferably where the cells are homozygous for the CFTR-ΔF508 mutation, are treated with drug candidate compounds, and the chloride efflux across the cell membrane is measured. For example, this technique (or variations thereof are described in Eidelman et al., supra.

(C) CFTR-Protein Binding Assay—Purified wild-type CFTR or mutant CFTR (e.g., CFTR-ΔF508) protein, or suitable portions thereof, can be utilized in vitro to identify compounds (i.e., drug candidates) that have the ability to bind CFTR in a protein-specific manner and with high affinity. Methods for the determination and quantitation of protein binding specificity and binding affinity are known in the art. The binding can be by any particular manner, but is most typically by non-covalent forces, such as hydrogen bonding, adsorption, absorption, metallic bonding, van der Waals forces, ionic bonding, or any combination thereof. In this case, portions of CFTR comprising the first nucleotide binding fold (NBF-1) are the preferred portions of CFTR to use in this type of assay. Compounds that bind with high affinity to CFTR, or a suitable portion of CFTR, are candidates for further development and testing.

Alternatively, the ability of a compound to bind to the wild-type and mutant CFTR proteins can be compared to identify candidate drugs, where compounds that bind preferentially to CFTR-ΔF508 compared to wild-type CFTR are also candidates for further development and testing.

The identification of compounds with binding specificity for CFTR protein, where the compound does not bind or binds with less affinity to other proteins, is a valuable indicator for drug screening. This is especially significant with regard to adenosine receptor proteins. Some compounds have been shown to bind the A1, A2 or A3-adenosine receptors, and/or antagonize the activity of those receptors. Compounds that antagonize adenosine receptors may not be ideal candidates for drug development, as those compounds may have toxic side effects when administered to a subject. However, it is not intended that the xanthine compounds finding use with the invention are limited to those compounds that do not bind or otherwise do not antagonize an adenosine receptor.

(D) Biochemical Activity Assays—Purified CFTR protein, or suitable portions of the protein, can be assayed in vitro for various biochemical activities in the absence and presence of drug candidate compounds. The induction or suppression of these activities in response to exposure to a test compound may be indicative that the compound has advantageous uses in the treatment of cystic fibrosis. For example, the various in vitro CFTR activities that can be measured include commencing or causing the aggregation of bovine chromaffin granules in the presence of CaCl₂, and commencing or causing the aggregation of liposomes. Such assays are described, for example, in U.S. Pat. No. 6,083,954.

It is not intended, however, that the present invention be limited to formulations comprising substituted xanthine compounds that strictly adhere to the above screening criteria. Furthermore, it is not intended that the invention be limited to any particular biochemical mechanism, as an understanding of the biochemical mechanisms underlying the properties of the invention is not necessary to make or use the invention. Thus, it is not necessary to have any understanding of the mechanism of the invention to make or use the invention.

It is intended, without limitation, that the substituted-xanthine compounds taught in U.S. Pat. Nos. 5,366,977, 5,877,179 and 6,083,954, the disclosures of which are hereby incorporated by reference in their entirety, find use in the novel drug formulations of the present invention.

Specific xanthine derivatives which find use in formulations of the present invention are listed below. However, it is not intended that the present invention be limited to those compounds listed below, as one of skill in the art immediately recognizes that variant molecules with structures related to the structures of the molecules listed below also find use with the invention.
1,3-dipropyl-7-methyl-8-cyclohexyl-xanthine
1,3-dipropyl-7-methyl-8-cyclopentyl-xanthine (DP-CPX)
1,3-diallyl-8-cyclohexyl-xanthine (DCHX)
1,3-dipropyl-8-cyclopentyl-xanthine (CPX)
1-propyl-8-cyclopentyl-xanthine
N,N-diallyl-8-cyclohexyl-xanthine (DAX)
1,3-diallyl-8-cyclohexyl-xanthine DCHX
1,3-dipropyl-7-methyl-8-cyclohexyl-xanthine
8-cyclohexyl caffeine (1,3,7-trimethyl-8-cyclohexyl-xanthine; CHC)
1,3-dimethyl-8-cyclohexyl-xanthine
1,3,7-trimethyl-8-(3-chlorostyrl)-xanthine (aka CSC)
theophylline
IBMX
xanthine amino congener (XAC)
KFM19
2-thio-CPX
KW-3902
CPT
caffeine

These compounds are described in various sources, including but not limited to, U.S. Pat. Nos. 4,548,818, 4,866,072, 5,032,593, 5,096,916, 5,366,977, 5,877,179 and 6,083,954; and references such as von der Leyen et al., Naunyn Schmiedebergs Arch. Pharmacol., 340(2):204-209 [1989]; Guay-Broder et al., Biochemistry 34(28):9079-9087 (1995); Müller et al., J. Med. Chem., 36:3341-3349 [1992]; Jacobson et al., Biochemistry 34:9088-9094 [1995]; Roomans, Exp. Opin. Invest Drugs 10(1):1-19 [2001]; Arispe et al., Jour. Biol. Chem., 273(10):5727-5734 [1998]; Rogers and Knox, Eur. Respir. Jour., 17:1314-1321 [2001]; Eidelman et al., Proc. Natl. Aced. Sc. USA 89:5562-5566 [1992]; and Ji et al., Jour. Receptor Research 12(2):149-169 [1992]. It is contemplated that substituted xanthine compounds described in the above U.S. patents and references can find use in the formulations of the invention.

Particularly preferred xanthine derivatives for use in the formulations of the present invention are 1,3-dipropyl-8-cyclopentylxanthine (CPX), N,N-diallylcyclohexylxanthine (DAX; synonymously, 1,3-diallyl-8-cyclohexylxanthine, DCHX), 1,3-dipropyl-7-methylcyclopenthylxanthine (DP-CPX), cyclohexylcaffeine (CHC), and xanthine amino congener.

Alternatively, or additionally, a pharmaceutically acceptable derivative of any of the compounds of the invention, or combinations of compounds, may be used in the present invention and inventive method, which provide yet another embodiment of the present invention. It is desirable that such a pharmaceutical derivative have equivalent therapeutic effectiveness in the context of the present inventive method of treatment.

In a most preferred embodiment, the compound 1,3-dipropyl-8-cyclopentyl-xanthine (CPX) is used in the formulations of the invention, which is in clinical development for the treatment of cystic fibrosis. CPX has numerous advantageous properties, including but not limited to, (a) activates chloride efflux from cell derived from a cystic fibrosis patient, (b) activates isolated CFTR channels in in vitro lipid bilayers, (c) binds to the NBF-1 region of CFTR, (d) its affinity for CFTR-ΔF508 NBF-1 is greater than the affinity of CPX for wild-type CFTR NBF-1, (e) enhances intracellular trafficking and maturation of CFTR-ΔF508, and (f) does not appear to display any mutagenicity or grossly apparent adverse side effects when oral doses are delivered to rat, guinea pig, mouse or dog model systems. Furthermore, CPX shows no apparent adverse side effects when oral doses are delivered to human subjects.

The present invention also encompasses all pharmaceutically acceptable salts of the foregoing compounds. One skilled in the art will recognize that acid addition salts of the presently claimed compounds may be prepared by reaction of the compounds with the appropriate acid via a variety of known methods. Alternatively, alkali and alkaline earth metal salts are prepared by reaction of the compounds of the invention with the appropriate base via a variety of known methods. For example, the sodium salt of the compounds of the invention can be prepared by reacting the compound with sodium hydride.

In the formulations of the present invention, the compounds of formula (I), including their derivatives and salts, are in pharmaceutically acceptable form. By pharmaceutically acceptable form is meant, inter alia, a pharmaceutically acceptable level of purity excluding normal pharmaceutical additives such as diluents and carriers, and including no material considered toxic at normal dosage levels. A pharmaceutically acceptable level of purity will generally be at least about 50% excluding normal pharmaceutical additives, preferably at least about 75%, more preferably at least about 90% still more preferably at least about 95%, and most preferably at least about 98%.

Preferably, the active compounds of formula (I) are sterilized before incorporation into the suspension formulations of the present invention. Sterilization may be performed, for example, by exposure to ethylene oxide before incorporation into the sterile vehicle (e.g., an oil).

Oil-based Suspensions of Substituted Xanthine Compounds for Oral Administration

Known formulations for the therapeutic delivery of substituted xanthine compounds utilize an aqueous delivery vehicle. In an effort to identify improved drug formulations displaying more advantageous pharmacokinetic properties, such as bioavailability and plasma half-life, the present inventors developed novel, oil-based formulations of substituted xanthine compounds suitable for oral delivery. More specifically, suspensions of xanthine derivatives in pharmaceutically acceptable oils with improved bioavailability and pharmacokinetic properties have been developed.

In tests that have led to the present invention, suspensions of CPX in corn oil were tested. In addition to CPX, this suspension formulation contained methylparaben and propylparaben as preservatives, and butylated hydroxytoluene (BHT) as an antioxidant. Details describing the preparation of the formulation are provided in Experimental EXAMPLE 1.

This corn oil-based formulation was used in side-by-side analyses with other CPX formulations, such a formulations comprising water, sodium carboxymethylcellulose (NaCMC; in homogenized or non-homogenized formulations), xanthan gum, and/or gelatin capsules, to test pharmacokinetic properties and bioavailability in rat and dog model systems. The corn oil-based formulations consistently showed statistically significant improved properties compared to other formulations.

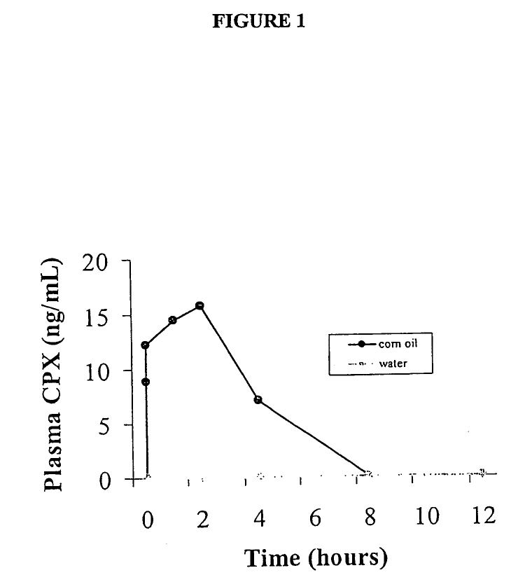

For example, as described in Experimental EXAMPLE 2, the blood plasma drug concentration of CPX was determined in rats following oral administration using either a water (i.e., aqueous) or a corn oil CPX formulation. As can be seen in FIG. 1, the differences in systemic CPX concentrations between the water and corn oil formulations is striking. The corn oil group displayed significant and sustained plasma CPX as long as 8 hours following drug delivery, while no individuals in the water vehicle group displayed any detectable plasma CPX.

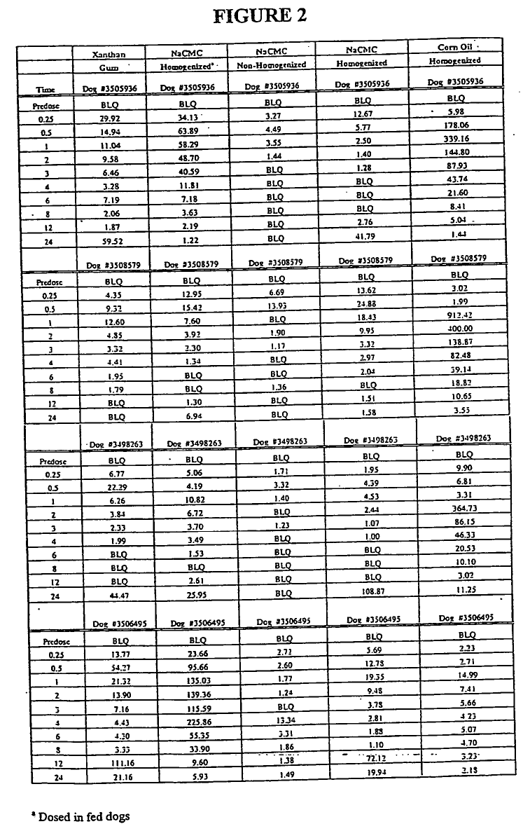

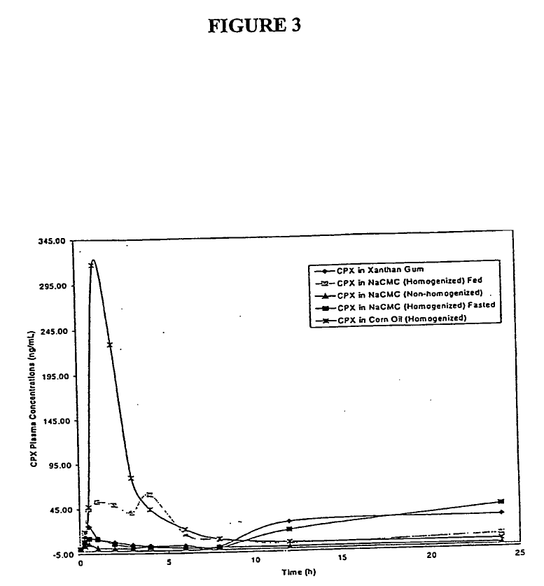

Experimental EXAMPLE 3 describes additional advantageous properties of corn oil formulations using a dog model system. Using this model system, the pharmacokinetics of CPX absorption were measured using various oral CPX formulations, including xanthan gum (homogenized), sodium carboxymethylcellulose [NaCMC] (homogenized), sodium carboxymethylcellulose [NaCMC] (non-homogenized), and corn oil (homogenized) formulations. In this experiment, the pharmacokinetic parameters Cₘₐₓ (maximum analyte concentration in the plasma, ng/ml), Tₘₐₓ (time of maximum analyte concentration in the plasma), and AUC (area under the curve for a defined period of time, where AUC is a measure of total systemic exposure expressed as ng-h/ml). The results of this experiment are summarized in FIGS. 2 and 3, and in TABLE 4. As can be seen in these FIGS. and TABLE, oral administration of the corn oil suspension formulation resulted in systemic CPX exposure which was at least two-fold greater than any other formulation tested. Based on plasma AUC(O-8) and Cₘₐₓ comparisons of the formulations tested, the oral bioavailability was highest with the corn oil formulation. Thus, the use of a corn oil CPX delivery formulation results in greater maximal drug concentration and greater overall systemic drug exposure compared to any other formulation tested.

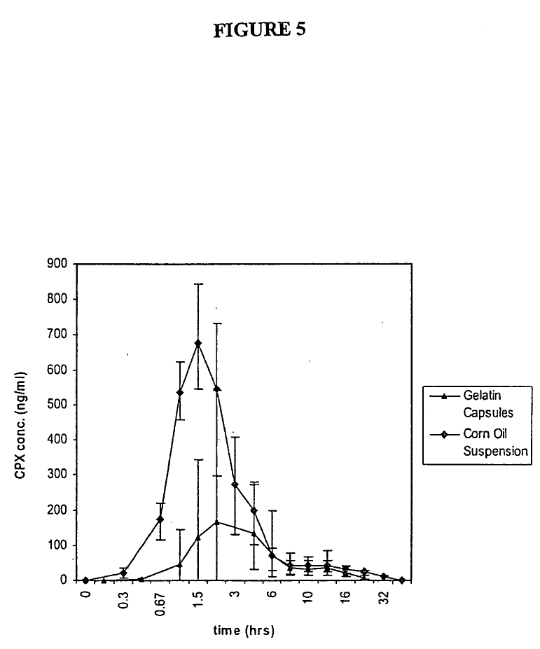

Human clinical studies were also undertaken to test the pharmacokinetic properties of an orally administered standard gelatin capsule CPX formulation or a novel corn oil CPX formulation. These pharamcokinetic properties were determined by monitoring the blood plasma CPX concentrations following oral administration of the formulations. These studies are described in Experimental EXAMPLE 4, and results are shown in FIG. 5 and TABLE 5. As can be seen in this data, the CPX concentrations in the subjects receiving the corn oil formulation reach a statistically significant higher level, and reach a Cₘₐₓ value much quicker compared to the concentration values in the subjects receiving the gelatin capsule CPX formulation. The corn oil formulation of CPX provided at least a two-fold greater maximal plasma CPX concentration, at least double total systemic CPX exposure (as measured by AUC_{inf}), and a longer half-life of the drug in the blood plasma (as measured by T_{1/2})

Although corn oil is used as an exemplary oral drug delivery vehicle, it is not intended that the present invention be limited exclusively to the use of corn oil as the drug delivery vehicle for substituted xanthine compounds. It is contemplated that numerous pharmaceutically acceptable natural or synthetic vegetable or animal oils find use as the oral drug delivery vehicle. Thus, oils suitable for use in the formulations of the present invention include vegetable oils, fish oils, animals fats and their partially or fully hydrogenated derivatives.

In a preferred embodiment, the delivery vehicle is a natural or synthetic pharmaceutically acceptable vegetable oil, comprising mono-, di, and/or trilgyceryl esters of saturated and/or unsaturated fatty acids. It is preferred that the oil be a glyceryl ester of a C₁₄-C₂₂ saturated and/or unsaturated fatty acids, triglycerides being particularly preferred.

Exemplary vegetable oils suitable for use as delivery vehicles in the formulations of the present invention include aceituno oil, almond oil, araehis oil, babassu oil, blackcurrant seed oil, borage oil, buffalo ground oil, candlenut oil, canola oil, caster oil, Chinese vegetable tallow oil, cocoa butter, coconut oil, coffee seed oil, corn oil, cottonseed oil, crambe oil, Cuphea species oil, evening primrose oil, grapeseed oil, groundnut oil, hemp seed oil, illipe butter, kapok seed oil, linseed oil, menhaden oil, mowrah butter, mustard seed oil, oiticica oil, olive oil, palm oil, palm kernel oil, peanut oil, poppy seed oil, rapeseed oil, rice bran oil, safflower oil, sal fat, sesame oil, shark liver oil, shea nut oil, soybean oil, stillingia oil, sunflower oil, tall oil, tea seed oil, tobacco seed oil, tung oil (China wood oil), ucuhuba, vernonia oil, wheat germ oil, hydrogenated caster oil, hydrogenated coconut oil, hydrogenated cottonseed oil, hydrogenated palm oil, hydrogenated soybean oil, hydrogenated vegetable oil, hydrogenated cottonseed and caster oil, partially hydrogenated soybean oil, partially hydrogenated soy and cottonseed oil, glyceryl tributyrate, glyceryl tricaproate, glyceryl tricaprylate, glyceryl tricaprate, glyceryl trundecanoate, glyceryl trilaurate, glyceryl trimyristate, glyceryl tripalmitate, glyceryl tristearate, glyceryl triarcidate, glyceryl trimyristoleate, glyceryl tripalmitoleate, glyceryl trioleate, glyceryl trilinoleate, glyceryl trilinolenate, glyceryl tricaprylate/caprate, glyceryl tricaprylate/caprate/laurate, glyceryl tricaprylate/caprate/linoleate, glyceryl tricaprylate/caprate/stearate, glyceryl tricaprylate/laurate/stearate, glyceryl 1,2-caprylate-3-linoleate, glyceryl 1,2-caprate-3-stearate, glyceryl 1,2-laurate-3-myristate, glyceryl 1,2-myristate-3-laurate, glyceryl 1,3-palmitate-2-butyrate, glyceryl 1,3-stearate-2-caprate; glyceryl 1,2-linoleate-3-caprylate.

Vegetable and non-vegetable oils, e.g., oils of animal origin, suitable for use in pharmaceutical formulations, as listed above, are readily available from commercial sources, including for example, Croda, Inc. and Croda International Plc. (East Yorkshire, UK), Abitec Corporation (London, UK and Columbus, Ohio), Research Plus, Inc. (South Plainfield, N.J.), Sigma (St. Louis, Mo.) and Larodan Fine Chemicals (Malmö, Sweden).

In some embodiments, a particularly noteworthy advantage of the invention is realized when the vegetable oil drug delivery formulation is used to deliver a substituted xanthine therapeutic compound that is water-sensitive and/or unstable in aqueous formulations, thereby protecting the drug from degradation.

In some embodiments, the vegetable oil formulations of the invention contain only vegetable oil and the xanthine drug. In other embodiments, the vegetable oil formulation comprises additional components such as preservatives (e.g., methylparaben and/or propylparaben), antioxidants (e.g., butylated hydroxytoluene; BHT), thickening agents, sweeteners (e.g. sucrose; lactose, fructose, glucose, mannitol, sorbitol, saccharin, cyclamate, acesulfam potassium, or taumatin), buffering agents, surfactants, solubilizers, flavorings (e.g., raspberry, strawberry and honey), odorants and/or colorants.

The pharmaceutical formulations of the present invention are provided in the form of oil-based suspensions, and intended for oral administration, optionally followed by the consumption of water. Accordingly, the concentration of the xanthine derivative in the formulation may vary within a wide range, and can preferably be up to the maximum amount that can be suspended, and further, the xanthine derivative has a homogenous uniform and optimal particle size that is instrumental in increasing bioavailability In general, the concentration of the xanthine derivative will be between about 0.1% and about 50% by weight, more preferably between about 1% and about 20% by weight, more preferably between about 1% and about 10% by weight.

A preferred pharmaceutical formulation herein has the following composition:

| | Concentration Range |
|---|---|
| Component | (% by weight) |
| vegetable oil | 85-95 |
| preservative | 0.0-0.5 |
| antioxidant | 0.0-0.5 |
| xanthine derivative | 1-10 |

In a particularly preferred embodiment, the formulations contain about 90-95% by weight corn oil, 4.0-8.0% by weight xanthine derivative, e.g., CPX, 0.05 to 0.15% by weight methylparaben and/or propylparaben, and optionally 0.05 to 0.15% butylated hydroxytoluene.

For optimal bioavailability, the mean particle size of the xanthine drug particle dispersed in the formulations should be less than about 100 microns. In one embodiment of the invention, the drug particles are preferably less than about 80 microns. In another embodiment, the drug particles are less than about 70 microns. In another embodiment, the drug particles are less than about 65 microns. However, it is not intended that the invention be limited to any particular drug particle size less than about 100 microns. It is contemplated that a range of particle sizes are equally suitable for use in the drug formulations. Furthermore, it is contemplated that different methods for drug crystallization will result in drug particles having differing and/or more advantageous properties. Different methods for drug crystallization can result in different optimal drug particle sizes to be used in the formulations. Thus, it is not intended that the invention be limited to any particular method for drug synthesis or crystallization, or drug particle size or size range.

If one round of homogenization does not provide the desired particle size and distribution, the homogenization process is repeated to ensure that the drug-particles are of the desired diameter. The small particle size of the active substance in the dispersions of the present invention as described also has the advantage of a slow rate of sedimentation of the suspended particles, which favorably affects the homogeneity of the liquid oral formulation of the active substance described and correspondingly ensures a high degree of accuracy in measuring the dose.

Preferably the formulations of the present invention are suitable for long term storage, and remain stable at room temperature for at least 6 months.

The formulations can be packaged into conventional containers, such as plastic or glass bottles conventionally used in the drug industry. The bottles are typically secured by a plastic screw cup, which is preferably child-resistant, have a label affixed to them, and might be accompanied by written directions for administration. Such articles of manufacture are within the scope of the invention.

The compound should be administered such that a therapeutically effective concentration of the compound is in contact with the affected cells of the body. The dose administered to a subject, particularly a human, in the context of the present invention should be sufficient to effect a therapeutic response in the animal over a reasonable period of time. The dose will be determined by the strength of the particular compound employed and the condition of the subject, as well as the body weight of the animal to be treated. The size of the dose also will be determined by the existence, nature, and extent of any adverse side effects that might accompany the administration of a particular compound.

The following examples serve to further illustrate the present invention and are not intended to limit the scope of the invention.

### 1. EXAMPLE 1

(a) CPX Formulations

This EXAMPLE describes the CPX formulations used in the present invention. Adequate solubility of CPX was unattainable in any of the solvents tested, even despite the use of co-solvents. This insolubility necessitated the use of the suspension and capsule formulations, as described below. One formulation was left non-homogenized to study the effect of homogenization on bioavailability.

Homogenization of CPX liquid formulations by mechanical means was used to attain uniform small particle size and a homogenous suspension. The drug/liquid vehicle mixtures were homogenized using a Brinkmann Polytron PT 6000 Homogenizer with the PT-DA 6045/6T generator at a homogenization speed setting of 10,800 rpm. Homogenization resulted in a mean particle diameter size of approximately 65 μm.

The formulations were prepared with various concentrations of CPX depending on the intended experiment, the intended model organism to be studied, or whether is would be used for human trials. The concentration of CPX in these formulations was confirmed using high performance liquid chromatography (HPLC) with mass spectrometric determination. The formulations were produced using Good Manufacturing Procedures (cGMPs). Quality assurance testing showed the formulations to be within the intended specification and sufficiently sterile. All formulations were stored at room temperature, and were demonstrated to be stable for at least 3 months. The formulations used were:

A) Sodium Carboxymethylcellulose [NaCMC] (homogenized), 2.175%. The mixture was homogenized to form a suspension, as described above.

B) Sodium Carboxymethylcellulose [NaCMC] (not homogenized), 2.175%

C) Xanthan Gum (homogenized), 0.4%.

The xanthan gum formulation used herein was a polysaccharide mixture containing glucose, mannose, potassium glucuronate, acetate and pyruvate. A drug delivery vehicle was formed by producing an aqueous 0.4% xanthan gum suspension, then adding CPX drug to a suitable concentration. The mixture was homogenized to form a suspension, as described above.

D) Corn Oil (homogenized)

This formulation used corn oil as the delivery vehicle for CPX drug. In addition to the CPX drug, this suspension formulation contained methylparaben and propylparaben as preservatives, and butylated hydroxytoluene (BHT) as an antioxidant.

Specifically, 9.853 kg of corn oil precombined with BHT was placed in a 20 liter mixing vessel and mixed at a speeds ranging from 444 to 750 RPM during the mixing process. To the stirring corn oil, 10.50 grams (0.1% by weight) of methylparaben was added, and stirred for 47 minutes until dissolved. Once dissolved, 6.3 grams (0.06% by weight) propylparaben were added to the corn oil mixture, and stirred for 73 minutes until dissolved. To this was then added 630 grams (6.0% by weight) CPX, and stirred for 40 minutes until the CPX was uniformly dispersed. This mixture was homogenized to form a suspension, as described above.

Placebo is supplied as corn oil solution containing methylparaben, propylparaben and butylated hydroxytoluene (BHT). Each dose of placebo matches the dose weight and volume utilized in the active portion of the corresponding non-placebo administration.

In one case, the CPX-corn oil formulation was packaged in a vessel convenient for dispensing the formulation to a subject. The vessel was a 2 ounce (capacity approximately 60 ml) amber glass bottle secured with a child-resistant plastic screw cap. The label applied to the container included the drug name, dosage strength, lot number, storage instructions, amount of suspension per container, and the manufacturer's name.

E) Gelatin Capsule (300 mg CPX unit dose)

F) Water Formulation

### 2. EXAMPLE 2

### CPX Absorption Profile in Rats Comparing Water and Corn Oil Drug Formulations

This EXAMPLE describes the pharmacokinetics of CPX absorption in rats following oral administration comparing two different liquid formulations containing the CPX drug, and demonstrates one of the advantageous properties of a corn oil CPX formulation over a water (i.e., aqueous) CPX formulation.

Experimental—This study was designed to determine the pharmacokinetics of CPX bioavailability in blood plasma following a single oral administration by oral gavage to male Sprague-Dawley rats. The CPX compound was suspended in either a corn oil or water formulation and administered once via oral gavage at 10 ml/kg of rat weight to two groups of male Sprague-Dawley rats. The two experimental groups are described in TABLE 1 below.

TABLE 1

| | CPX Dosage | | Number of | Animal |
|---|---|---|---|---|
| Group | (mg/kg) | Vehicle | Individuals | Identification Nos. |
| 1 | 1.4 | corn oil | 10 | 17869-17878 |
| 2 | 1.4 | water | 10 | 17879-17888 |

There was no mortality or signs of morbidity noted at any time during the course of this experiment. Blood was collected from the first five animals in each group at 0.25, 1, 4 and 12 hours following dosing, and from the second five animals in each group at 0.5, 2, 8 and 24 hours following dosing. The rats were not fasted prior to blood collection. Approximately 0.4 ml of whole blood was collected from each animal into heparinized tubes via puncture of the orbital sinus under 70% CO₂/30% O₂ anesthesia. Approximately 0.2 ml of plasma was separated by centrifugation and analyzed for CPX concentration using high performance liquid chromatography (HPLC) with mass spectrometric detection. Following the completion of blood collection, all surviving animals were euthanized by carbon dioxide overdose.

Results/Conclusions—The results of this plasma CPX concentration analysis are shown in TABLE 2 below. In TABLE 2, CPX quantitation is shown in ng/ml. The ten individuals receiving the corn oil CPX formulation are shown in the top rows, while the ten individuals receiving the water CPX formulation are shown in the bottom rows.

TABLE 2

| Animal ID | Time in hours | | | | | | | |
|---|---|---|---|---|---|---|---|---|
| No. | 0.25 | 0.5 | 1 | 2 | 4 | 8 | 12 | 24 |
| 17869 | 18.46 | — | 23.78 | — | 21.17 | — | BLQ | — |
| 17870 | BLQ | — | BLQ | — | 15.05 | — | BLQ | — |
| 17871 | 12.33 | — | 18.07 | — | 27.87 | — | BLQ | — |
| 17872 | 13.67 | — | 19.30 | — | 14.92 | — | BLQ | — |
| 17873 | BLQ | — | BLQ | — | BLQ | — | BLQ | — |
| 17874 | — | 13.08 | — | 18.02 | — | BLQ | — | BLQ |
| 17875 | — | 10.31 | — | 12.52 | — | BLQ | — | BLQ |
| 17876 | — | 11.91 | — | 17.60 | — | 12.49 | — | BLQ |
| 17877 | — | 12.85 | — | 11.09 | — | 10.31 | — | BLQ |
| 17878 | — | BLQ | — | 13.34 | — | 12.62 | — | BLQ |
| 17879 | BLQ | — | BLQ | — | BLQ | — | BLQ | — |
| 17880 | BLQ | — | BLQ | — | BLQ | — | BLQ | — |
| 17881 | BLQ | — | BLQ | — | BLQ | — | BLQ | — |
| 17882 | BLQ | — | BLQ | — | BLQ | — | BLQ | — |
| 17883 | BLQ | — | BLQ | — | BLQ | — | BLQ | — |
| 17884 | — | BLQ | — | BLQ | — | BLQ | — | BLQ |
| 17885 | — | BLQ | — | BLQ | — | BLQ | — | BLQ |
| 17886 | — | BLQ | — | BLQ | — | BLQ | — | BLQ |
| 17887 | — | BLQ | — | BLQ | — | BLQ | — | BLQ |
| 17888 | — | BLQ | — | BLQ | — | BLQ | — | BLQ |

Quantitation in ng/ml.
BLQ = Below Limit of Quantitation
— = No Sample Expected

Rat plasma CPX concentration as a function of time (in hours) using the two formulations was measured. Each rat in this experiment (n=10) received a 1.4 mg/kg CPX dose, which was equivalent to a 100 mg dose. Ten rats were used at each time point to generate a mean CPX concentration value. The results of this experiment are depicted graphically in FIG. 1.

As can be seen in TABLE 2 above and in FIG. 1, the differences in systemic CPX concentrations between the water and corn oil formulations is striking. The corn oil group displayed between 10 and 27 ng/ml plasma CPX as long as 8 hours following drug delivery, while no individuals in the water vehicle group displayed any detectable plasma CPX. Thus, the corn oil vehicle formulation provided great benefit over the aqueous vehicle formulation as measured by CPX bioavailability.

### 3. EXAMPLE 3

### CPX Absorption Profiles Comparing Various CPX Drug Formulations in Dogs

This EXAMPLE describes the pharmacokinetics of CPX absorption in dogs as measured in blood plasma following oral administration comparing four different CPX liquid formulations, and demonstrates one of the advantageous properties of a corn oil CPX formulation over other CPX formulations.

Experimental—This study was designed to determine the relative bioavailability of a single oral CPX dose when administered by gavage to male Beagle dogs. The CPX dosages was administered in a single 30 mg/kg oral dose in four different suspension formulations. These suspension formulations were:
1) xanthan gum (homogenized),
2) sodium carboxymethylcellulose [NaCMC] (homogenized),
3) sodium carboxymethylcellulose [NaCMC] (non-homogenized), and
4) corn oil (homogenized).

Each formulation contained CPX at a nominal concentration of 60 mg/gram of suspension. Nominal doses of 30 mg/kg animal weight were administered for each formulation, and were administered gravimetrically at 0.5 g/kg (approximately 6.0 g/dog) by gavage. A total of four male beagle dogs were used in the study. The analysis of each formulation comprised data from four dogs (n=4). A combination of naive and non-naive dogs were used, and a one week “washout period” was maintained between each formulation trial. Animals were fasted overnight prior to each dose administration. In one experiment using the NaCMC homogenized formulation, animals were inadvertently not fasted (data indicated below).

Whole blood samples were collected (approx. 3 ml/sample) by jugular venipuncture into sodium heparin-containing collection tubes. Samples were collected at times 0 (predose) and at 0.25, 0.5, 1, 2, 3, 4, 6, 8, 12 and 24 hours after dosing. The whole blood was centrifuged to isolate plasma, and concentrations of CPX in the plasma at these time intervals were determined using high performance liquid chromatography (HPLC) with mass spectrometric detection, with a lower quantitation limit of 1 ng/ml.

The following pharmacokinetic parameters were determined for each experimental CPX formulation:
Cₘₐₓ—maximum analyte concentration in the plasma, ng/ml
Tₘₐₓ—time of maximum analyte concentration in the plasma
T_{1/2}—terminal half-life of the drug
AUCₗₐₛₜ—area under the curve (AUC) from time 0 to the last measurable concentration. The AUC is a measure of total systemic exposure over a defined time interval. Expressed as ng·h/ml AUC_{(0∞)}—area under the curve from time 0 to infinity (also written AUC_{inf} or AUC_{∞})

Results/Conclusions—No adverse effects were apparent after oral administration of any of the CPX formulations. The CPX concentration values (ng/ml) at each time point were determined for each dog in this study are shown in FIG. 2. Time is shown in hours, and each of the four dogs is indicated by its Identification Number. It was observed that measured peak concentrations of CPX in the plasma occurred within 5 hours of oral administration dosing.

The data in FIG. 2 was condensed by determining the mean CPX plasma concentration (ng/ml) for the group of dogs receiving the same drug formulation (n=4). This mean was calculated for each time point. The mean CPX concentration values are summarized in TABLE 3, below. Also included are a single set of data for dogs that received the homogenized NaCMC formulation that were inadvertently not-fasted (i.e., the dogs were fed).

TABLE 3

| | Plasma CPX Concentration (ng/ml ± SEM^{a}) | | | | |
|---|---|---|---|---|---|
| | | | NaCMC | | NaCMC |
| time | xanthan gum | NaCMC | (non- | corn oil | (homogenized, |
| (hours) | (homogenized) | (homogenized) | homogenized) | (homogenized) | fed) |
| 0 | BLQ^{b} | BLQ | BLQ | BLQ | BLQ |
| 0.25 | 13.70 ± 11.53 | 8.48 ± 5.61 | 3.60 ± 2.16 | 5.28 ± 3.48 | 18.95 ± 12.67 |
| 0.5 | 25.21 ± 20.09 | 11.95 ± 9.37 | 6.08 ± 5.29 | 47.39 ± 87.14 | 44.79 ± 42.68 |
| 1 | 12.81 ± 6.28 | 11.20 ± 8.92 | 1.68 ± 1.46 | 317.47 ± 426.08 | 52.96 ± 59.44 |
| 2 | 8.04 ± 4.64 | 5.82 ± 4.53 | 1.14 ± 0.81 | 229.23 ± 186.06 | 49.67 ± 63.20 |
| 3 | 4.82 ± 2.35 | 2.36 ± 1.39 | BLQ | 79.65 ± 55.05 | 40.55 ± 53.08 |
| 4 | 3.53 ± 1.16 | 1.69 ± 1.44 | NC | 44.20 ± 31.98 | 60.62 ± 110.25 |
| 6 | 3.36 ± 3.10 | BLQ | NC | 21.58 ± 13.93 | 16.24 ± 26.23 |
| 8 | 1.79 ± 1.37 | NC^{c} | BLQ | 10.51 ± 5.98 | 9.66 ± 16.23 |
| 12 | 28.26 ± 55.28 | 19.10 ± 35.37 | BLQ | 5.48 ± 3.56 | 3.93 ± 3.82 |
| 24 | 31.29 ± 26.16 | 43.05 ± 46.86 | BLQ | 4.60 ± 4.52 | 10.01 ± 10.92 |

^{a}SEM = standard error of the mean
^{b}BLQ = Below Limit of Quantitation
^{c}NC = mean value not calculated (>50% of individual concentrations were BLQ)

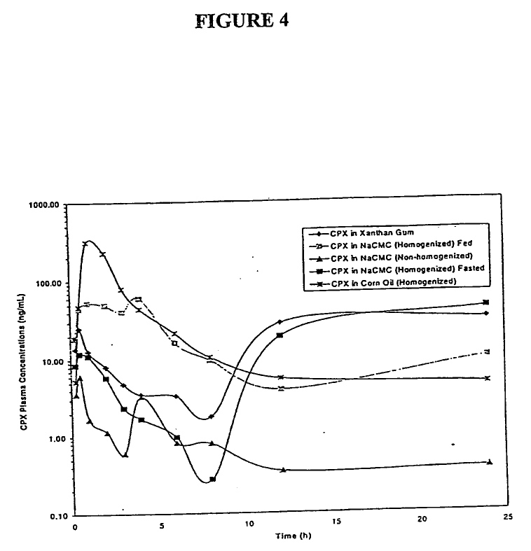

This data in TABLE 3 above is depicted graphically in FIGS. 3 and 4. FIG. 3 plots the mean CPX plasma concentration (ng/ml) of each fasted dog group versus time (in hours), for each formulation, on a linear axis. Each data point on this graph represents a mean value derived from four animals (n=4). Also included are a single set of data for dogs that received the homogenized NaCMC formulation that were inadvertently not-fasted (i.e., the dogs were fed). FIG. 4 shows this same data, but on a semilogarithmic concentration scale. As can clearly be seen in both of these plots, the CPX concentration in the plasma is strikingly higher when the corn oil CPX formulation was used, as compared to any of the other formulations.

Pharmacokinetic analysis of this same data was also undertaken. The results of this analysis are shown in TABLE 4 below. The standard error of the mean is also shown.

TABLE 4

| | Formulation | | | | |
|---|---|---|---|---|---|
| | | | | | NaCMC |
| | xanthan gum | NaCMC (non- | NaCMC | corn oil | (homogenized, |
| parameter | (homogenized) | homogenized) | (homogenized) | (homogenized) | non-fasted) |
| Cₘₐₓ (ng/ml)^{a} | 29.8 ± 17.8 | 8.75 ± 5.62 | 15.4 ± 8.79 | 408 ± 372 | 79.0 ± 101 |
| | (51.4 ± 44.6) | | (22.8 ± 15.4) | | (82.8 ± 97.7) |
| tₘₐₓ (hours)^{a} | 0.56 ± 0.32 | 1.38 ± 1.75 | 0.69 ± 0.38 | 1.25 ± 0.50 | 1.50 ± 1.68 |
| | (9.4 ± 11.1) | | (12.4 ± 13.4) | | (7.25 ± 11.3) |
| AUC(0-8 h) | 48.7 ± 25.6 | 13.3 ± 13.3 | 27.0 ± 20.4 | 687 ± 578 | 284 ± 406 |
| (ng · h/ml) | | | | | |
| AUC(0-24 h) | 398 ± 506 | 19.2 ± 25.0 | 276 ± 332 | 779 ± 615 | 395 ± 449 |
| (ng · h/ml) | | | | | |

^{a}Cₘₐₓ and tₘₐₓ values in parenthesis indicate parameters calculated with 0-24 hour data (including any elevated terminal concentration-time points).

From TABLE 4 above, it can be seen that oral administration of the corn oil suspension formulation resulted in systemic CPX exposure which was at least twofold greater than any other formulation tested. Based on plasma AUC(0-8) and Cₘₐₓ comparisons of the formulations tested, the oral bioavailability was highest with the corn oil formulation followed in decreasing order by xanthan gum, NaCMC (homogenized), and lastly, NaCMC (non-homogenized).

CPX systemic exposure following administration of the NaCMC (homogenized) formulation was approximately 20-fold greater in non-fasted dogs compared to fasted dogs.

In dogs, Cₘₐₓ was 0.4 μg/ml following a 30 mg/kg dose of CPX in the corn oil formulation. This is in contrast to 0.1 μg/ml observed in previous dog studies using a methyl cellulose formulation.

Thus, the use of a corn oil CPX delivery formulation results in greater maximal drug concentration and greater overall systemic drug exposure compared to any other formulation tested.

### 5. EXAMPLE 4

### CPX Absorption Profile in Humans Comparing Gelatin Capsule and Corn Oil Formulations

This EXAMPLE describes the pharmacokinetics of CPX absorption in humans following oral administration of two different drug formulations, namely, a gelatin capsule formulation and a corn oil formulation, and demonstrates the advantageous properties of a corn oil CPX formulation over a standard gelatin capsule formulation.

Expermental—CPX was supplied in two different formulations. These were a suspension in corn oil containing 60 mg of CPX per gram of the suspension, as described in EXAMPLE 1, and a hard gelatin capsule.

A single 300 mg oral dose of CPX was administered to the subjects in this experiment. The 300 mg CPX dose was contained in either the corn oil formulation (i.e., 5 ml dosages of 60 mg/ml formulation) or a hard gelatin capsule formulation. The gelatin capsule formulation was administered to cystic fibrosis patient subjects (n=4), and the corn oil formulation was delivered to normal male subjects (n=3).

Blood samples were collected for determination of plasma CPX concentration. For each group, 10 ml samples were collected by indwelling catheter or by venipuncture from the appropriate vein into sodium heparin collection tubes. For each individual, whole blood samples were collected predose (t-0), 20 minutes, 40 minutes, and 1, 1.5, 2, 3, 4, 6, 8, 10, 12, 16, 24, 32 and 48 hours following administration. The blood samples were centrifuged to isolate plasma, and concentrations of CPX in the plasma at these time intervals were determined using high performance liquid chromatography (HPLC) with mass spectrometric detection, with a lower quantitation limit of 1 ng/ml. Using these CPX concentration values, pharmacokinetic analysis was conducted.

Results/Conclusions—No adverse effects were reported after oral administration of either CPX formulation. A graphical representation of the plasma CPX concentrations that were measured in this experiment are provided in FIG. 5. As can be seen in this FIG., the CPX concentrations in the subjects receiving the corn oil formulation reach a statistically significant higher level, and reach a Cₘₐₓ value much quicker compared to the concentration values in the subjects receiving the gelatin capsule CPX formulation.

Results of the pharmacokinetic analysis are shown in TABLE 5, below. Standard deviation values of the means are also indicated.

TABLE 5

| | Single 300 mg CPX Dose | |
|---|---|---|
| | | Corn Oil Formulation |
| | CF patients (n = 4) | Normal Males (n = 3) |
| Cₘₐₓ (mean), ng/ml | 259 ± 191 | 676 ± 154 |
| Cₘₐₓ range | 144-543 | 544-845 |
| AUC_{inf} (mean), | 1217 ± 824 | 2621 ± 506 |
| ng · h/ml | | |
| AUC_{inf} range | 581-2423 | 2061-3043 |
| T_{1/2} (mean), hours | 8.5 ± 4.8 | 13.7 ± 5.3 |

Thus, as can be seen in TABLE 5, the corn oil formulation of CPX provided at least a two-fold greater maximal plasma CPX concentration, at least double total systemic CPX exposure (as measured by AUC_{inf}), and a longer half-life of the drug in the blood plasma (as measured by T_{1/2}).

### 7. EXAMPLE 5

(a) CPX Phamacokinetic Clinical Studies in Humans Using a Corn Oil Drug Formulation

This EXAMPLE provides a protocol for the further analysis of safety and pharmacokinetic behavior of CPX when administered to humans in a corn oil vehicle for oral administration. The corn oil suspension used in this study is the same as described in EXAMPLE 1. The goals of this protocol are
(1) to define the CPX corn oil oral suspension dose which achieves a maximal AUC of approximately 3275 ng·h/ml, and simultaneously is safe and tolerable,
(2) to characterize the safety and tolerance of CPX corn oil formulations required to achieve an AUC up to approximately 4500 ng·-h/ml,
(3) to compare the concentration versus time profiles of plasma CPX concentration following administration of a corn oil-CPX formulation following a high-fat breakfast compared to administration under fasted conditions, and
(4) to define safe and tolerable corn oil-CPX dosage regimens that result in steady-state trough CPX levels that exceed 300 ng/ml.

Parts (1), (2) and (3) of this study are conducted as a single-blind, randomized, placebo controlled single dose, pharmacokinetically guided dose escalation study. Administration of the corn oil formulations (CPX-containing or placebo) is directly into the subjects mouth vial an oral syringe, followed by the ingestion of 240 ml of water. Different groups of four subjects are to receive either a placebo (n=1) or one of up to six doses of CPX corn oil suspension (n=3) under fasted conditions targeted to achieve a maximum AUC up to approximately 4500 ng·hr/ml. Upon completion of the highest dose group, a different group of four subjects repeats one of the doses given previously, in order to increase the number of subjects for analysis. In addition, a different group of four subjects is administered either placebo (n=1) or one of the doses of CPX oral suspension (n=3) given previously with a high-fat breakfast. Up to eight groups of four subjects participate in this phase of the study.

The first dose used in the study is 30 mg (0.5 grams of an oral suspension containing 60 mg of CPX per gram of the suspension). Provided that no dose-limiting adverse effects are observed, dose escalation is pharmacokinetically guided. If the AUC for the 30 mg dose is less than or equal to 1000 ng·hr/ml (approximately one-third of the maximum AUC observed as safe and tolerable in the previous Phase I single dose study), the second dose is 100 mg. Otherwise, the second dose is selected based on predicted dose to achieve an AUC of approximately 3275 ng·hr/mL. If the second dose results in AUC less than 3275 ng·hr/mL, the third dose is selected to achieve an AUC of approximately 3275 ng·hr/ml. Subsequent dose level(s) to achieve an AUC of up to approximately 4500 ng·hr/ml is selected primarily based on pharmacologic effects or adverse events; however, doses are selected to produce no more than a 33% increase in AUC. Dose groups are evaluated in 7-14 day intervals upon the condition that the dose given to the previous dose group is deemed safe and tolerable.

If dose limiting adverse effects are observed in one or more CPX-treated subject at a given dose, the next group of four subjects is administered the same dose. Should dose-limiting adverse effects not be observed in the additional dose group, dose escalation resumes. If however, dose-limiting adverse effects are observed in one or more CPX-treated subjects in the additional dose group, dose escalation is discontinued and an optional step-down dose equal to the mid-point between the highest dose and the previous tolerated dose is given. At any time during the study, an individual must be withdrawn from the study in the event that the subject experiences an intolerable treatment-emergent adverse event as determined by the investigator or subject. Should an intolerable treatment-emergent adverse event or a serious adverse event attributed to the study drug by the investigator as possible, probable or definite, occur in one or more subject at any time during a dose group, the study sponsor and manager jointly determine whether to discontinue the dose group. The dose escalation is adjusted from the original plan upon discussion with the study sponsor.

Part 4 of the study (i.e., determination of safe and tolerable corn oil-CPX dosage regimens that result in steady-state trough CPX levels that exceed 300 ng/ml) is conducted as a single-blind, randomized, placebo-controlled, multiple dose study. Different groups of eight subjects receive either placebo (n=2) or one of two dosage regimens of CPX oral corn oil suspension (n=6). The first dosage regimen is selected to achieve steady state trough plasma CPX concentrations of 300 ng/ml, assuming that the predicted AUC does not exceed values deemed safe and tolerable in the first phase of the study. The second dosage regimen is selected to achieve steady trough plasma CPX concentrations of 600 ng/ml, assuming that the predicted AUC does not exceed values deemed safe and tolerable in the first phase of the study. A new dosage regimen will not be evaluated until the previous dosage regimen is deemed safe and tolerable.

At any time during the study, an individual must be withdrawn from the study in the event that the subject experiences an intolerable treatment emergent adverse event as determined by the investigator or subject. Should dose limiting adverse effects occur in two or more CPX-treated subjects on a given dosage regimen, or an intolerable treatment-emergent adverse effect or a serious adverse effect attributed to the study drug by the investigator as possible, probable or definite, occur in one or more subject at any time during a dose group, the study sponsor and manager will jointly determine whether to discontinue the dose group. The selection of dosage regimen may be adjusted from the original plan upon discussion with the study sponsor.

For the pharmacokinetic analysis in parts (1), (2) and (3), blood samples will be collected prior to dose and 20 and 40 minutes and 1, 1.5, 2, 3, 4, 6, 8, 10, 12, 16, 24, 32 and 48 hours following administration of the single oral dose. CPX concentration in the blood plasma will be determined for each sample. For the pharmacokinetic analysis in part (4), blood samples will be collected prior to the first and last dose and 20 and 40 minutes and 1, 1.5, 2, 3, 4, 6, 8, 10, 12, 16, 24, 32 and 48 hours following the last dose for determination CPX concentration in the blood plasma. Predose samples will be collected prior to the first dose given on days 4, 5 and 6. Additional samples are collected after the first dose given on day 4 at 2, 4, 6, 10 and 12 hours post-dose. Pharmacokinetic data for each CPX dose will be summarized using descriptive statistics.

All of the references identified herein, including patents, patent applications, and publications, are hereby incorporated by reference in their entireties.

While the invention has been described with an emphasis upon preferred embodiments, it will be obvious to those of ordinary skill in the art that variations in the preferred method, compound, and composition can be used and that it is intended that the invention can be practiced otherwise than as specifically described herein. Accordingly, this invention includes all modifications encompassed within the spirit and scope of the invention as defined by the following claims.

What is claimed is:

8. A liquid pharmaceutical formulation suitable for oral administration comprising an effective amount of a therapeutically active xanthine derivative, substituted xanthine, or pharmaceutically acceptable salt of said xanthine derivative or said substituted xanthine, having the formula: 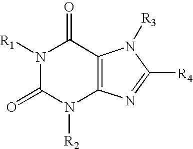
(I), wherein
R₁ and R₂ are the same or different and are C(1-6)alkyl or C(1-6)alkenyl, or hydrogen; R₃ is C(1-6)alkyl or hydrogen, and R₄ is C(48)cycloalkyl, aryl or hydrogen, wherein at least one of R₁, R₂ and is other than hydrogen, in admixture with a pharmaceutically acceptable oil.

9. The formulation of claim 1, wherein said formulation is a solution.

10. The formulation of claim 1, wherein said xanthine derivative, substituted xanthine, or pharmaceutically acceptable salt of said xanthine derivative or said substituted xanthine, is hydrophobic.

11. The formulation of claim 1, wherein said formulation is a suspension.

12. The formulation of claim 4, wherein said suspension is substantially homogenous.

13. The formulation of claim 4, wherein said therapeutically active xanthine derivative, substituted xanthine, or pharmaceutically acceptable salt of said xanthine derivative or said substituted xanthine, is in the form of particles having a mean diameter less than about 100 microns.

14. The formulation of claim 1 further comprising a pharmaceutically acceptable preservative.

15. The formulation of claim 1 further comprising a pharmaceutically acceptable antioxidant.

16. The formulation of claim 1 wherein said pharmaceutically acceptable oil is a vegetable oil.

17. The formulation of claim 9 wherein said vegetable oil is selected from the group consisting of corn oil, almond oil, coconut oil, cottonseed oil, mustard seed oil, olive oil, palm oil, peanut oil, safflower oil, sesame oil, soybean oil, sunflower oil, and partially or fully hydrogenated derivatives of said oils.

18. The formulation of claim 10 wherein said vegetable oil is corn oil.

19. The formulation of claim 1 wherein in said formula (I) R₁ and R₂ are the same or different and are C(1-6)alkyl or C(1-6)alkenyl; R3 is C(1-6)alkyl or hydrogen, and R4 is C(4-8) cycloalkyl.

20. The formulation of claim 1 wherein in said formula (I) R₁ and R₂ are the same and are methyl or allyl, R₃ is ethyl, cyclopropylmethyl or hydrogen, and R4 is cyclohexyl, provided that R₁ is allyl when R₃ is hydrogen, and R₁ is methyl when R₃ is ethyl or cyclopropylmethyl.

21. The formulation of claim 1 wherein in said formula (I) R₁ and R₂ are both methyl, R₃ is ethyl, cyclopropylmethyl, and R₄ is cyclohexyl.

22. The formulation of claim 1 wherein in said formula (I) R₁ and R₂ are allyl, R₃ is hydrogen, and R₄ is cyclohexyl, cyclohexylmethyl, or cycloheptyl.

23. The formulation of claim 1 wherein in said formula (I) R₁ is methyl, R₂ is allyl, R₃ is cyclopropylmethyl or ethyl, and R₄ is cyclohexyl.

24. The formulation of claim 1 wherein in said formula (I) R₁ and R₂ are the same or different, and are methyl, propyl, allyl or hydrogen; R₃ is methyl or hydrogen, and R₄ is cyclohexyl or cyclopentyl.

25. The formulation of claim 1 wherein said substituted xanthine of formula (I) is selected from the group consisting of 1,3-dipropyl-cyclopentylxanthine (CPX), 1,3-diallyl-cyclohexylxanthine (DAX/DCHX), 1,3-dipropyl-7-methylcyclopenthylxanthine (DP-CPX), cyclohexylcaffeine (CHC), and xanthine amino congener (XAC).

26. The formulation of claim 18 wherein said substituted xanthine of formula (I) is 1,3-dipropyl-8-cyclopentylxanthine (CPX).

27. A method for the activation of ion efflux in ion efflux deficient cells, comprising contacting said cells with an effective amount of a liquid pharmaceutical formulation according to one of claims 1 through 19.

28. The method of claim 20 wherein said cells are cystic fibrosis (CF) cells.

29. The method of claim 21 wherein said cells have the CFTR-ΔF508 mutation.

30. A method for the treatment of a chronic obstructive airway disorder, comprising administering to a subject in need a therapeutically effective amount of a liquid pharmaceutical formulation according to one of claims 1 through 19.

31. The method of claim 23 wherein said chronic obstructive airway disorder is characterized by defective ion transport associated with reduced or abnormal CFTR activity.

32. The method of claim 24 wherein said disease or condition is cystic fibrosis.

33. An article of manufacture comprising:
a) a container;
b) a liquid pharmaceutical formulation according to one of claims 1 through 19 within said container; and
c) directions for administration of said formulation for the treatment of a disease or condition characterized by defective ion transport associated with reduced or abnormal CTFR activity.
